(19) United States
(12) Patent Application Publication
(10) Pub. No.: US 2012/0177782 A1
(43) Pub. Date: Jul. 12, 2012

(54) SOY MILK FERMENTATION

(75) Inventors: Franciscus Adrianus Maria De Bok, Wageningen (NL); Patrick Wilhelmus Maria Janssen, Ede (NL); Jan Sikkema, Zeegse (NL); Eilt Johannes Smid, Wageningen (NL)

(73) Assignee: DSM IP ASSETS B.V., Heerlen (NL)

(21) Appl. No.: 13/394,772

(22) PCT Filed: Sep. 10, 2010
(86) PCT No.: PCT/NL2010/050572

§ 371 Date: Mar. 19, 2012

Related U.S. Application Data
(60) Provisional application No. 61/241,117, filed on Sep. 10, 2009.

(30) Foreign Application Priority Data
Sep. 10, 2009 (EP) 09169906.6

(51) Int. Cl. A23L 1/20; C12N 1/20; C12Q 1/02

(52) U.S. Cl. 426/46; 435/29; 435/252.9; 426/61

(57) ABSTRACT

The present invention relates to a method for preparing a soy-based fermented food product using L. delbrueckii and a strain capable of stimulating growth of L. delbrueckii, and a food product obtainable by such method. The invention also provides for a culture comprising said strains, and the use thereof for preparing said fermented food product.

### FIELD OF THE INVENTION

The present invention is in the field of fermentation of an aqueous medium comprising soy protein as main protein constituent, in particular to obtain soy yogurt. The invention provides for a method for production of a soy-based fermented food product, and a soy-based fermented food product thus obtained. The invention also relates to new cultures for the preparation of such soy-based fermented food product.

### BACKGROUND

According to the Codex Alimentarius (FAO/WHO 1977), yogurt is milk fermented with symbiotic cultures of Lactobacillus delbrueckii subsp. bulgaricus and Streptococcus thermophilus. Also, Lactobacillus delbrueckii subsp. lactis is sometimes used in the preparation of dairy yogurt.

In soy yogurts the main source of protein is not milk, but soy. Soy yogurts are usually fermented with yogurt culture, i.e., symbiotic cultures of Lactobacillus bulgaricus (Lactobacillus delbrueckii subsp. bulgaricus) and Streptococcus thermophilus, optionally in conjunction with further lactic acid bacteria.

Soybeans are less expensive and more abundant than bovine milk, and do not contain cholesterol. Yogurt is one of the fastest growing cultured dairy product in the Western world. Soy yogurt may command a market on the strength of being cholesterol-free. Thus, the economic incentive exists to develop a soy yogurt, in which soy can be utilized as a major ingredient. The basis for such soy yogurt may be soy milk, originating from the soybean. The major carbohydrates in soybeans are sucrose, raffinose and stachyose. The main problem limiting widespread consumption of soybeans and derivatives thereof such as soy yogurt is their objectionable beany flavor and high levels of the oligosaccharides raffinose and stachyose, which can lead to flatulence when consumed in considerable amounts.

Lactobacillus delbrueckii subsp. bulgaricus (hereinafter also referred to as “Lactobacillus delbrueckii bulgaricus”, “Lactobacillus bulgaricus” or “L. bulgaricus”) generally shows limited outgrowth in soy milk compared to bovine milk. This affects the properties of a fermented soy product which has been fermented using Lactobacillus delbrueckii subsp. bulgaricus. The present inventors have now found that certain Lactobacillus plantarum strains improve outgrowth of Lactobacillus delbrueckii subsp. bulgaricus in soy protein-comprising media, thereby improving the yogurt-type product that is obtained by fermenting soy milk using Lactobacillus delbrueckii subsp. bulgaricus and Streptococcus thermophilus compared to such product obtained by fermenting in the absence of such Lactobacillus plantarum strains.

L. delbrueckii subsp. bulgaricus degrades sucrose, which is one of the main sugar constituents present in soy milk, poorly. Without wishing to be bound by theory, it is hypothesized that the Lactobacillus plantarum strains affecting growth of L. delbrueckii subsp. bulgaricus are capable of breaking down sucrose, raffinose and/or stachyose molecules into substrates allowing growth of L. delbrueckii subsp. bulgaricus.

### SUMMARY OF THE INVENTION

A combinatorial screening approach to identify strain-dependent traits related to mixed culture performance in natural substrates was developed and used to screen a collection of L. plantarum strains for 2- and 3-species mixed culture growth with Streptococcus thermophilus CNRZ1066 and Lactobacillus delbrueckii subsp. bulgaricus ATCC-BAA365 in milk and soymilk. This revealed a strain dependent stimulatory effect of L. plantarum B1839, B2484, and B2485 on L. bulgaricus in soymilk, resulting in a mutualistic relationship independent of the presence of S. thermophilus. While in 38 out of 40 mixed cultures with L. plantarum, the levels of L. bulgaricus were below the detection limit (500 CFU/ml) after 4 rounds of fermentation in soymilk (5-ml static batch fermentations for 24 h at 37° C.), strain B1839 stimulated L. bulgaricus over a 100-fold when compared to the corresponding monoculture, yielding nearly 10⁸ CFU/ml. The effect was also observed in the 3-species mixed cultures further including S. thermophilus yielding a 3-species stable culture. The stimulatory effect could be reproduced in 1 L stirred batch fermentations which were performed to study the population dynamics of all possible combinations of L. plantarum strain B1839 in soymilk. Clearly strain B1839 stimulated growth of L. bulgaricus in natural soymilk (no additional sugars added) and was thereby able to confer yoghurt-like properties to fermented soymilk products. In 3-species mixed cultures, also strains B2484 and B2485 had a stimulatory effect on L. bulgaricus in soy milk.

Thus, in a first aspect the present invention provides for a method for preparing a fermented soy product, said method comprising the step of fermenting an aqueous medium comprising 0.25-10% (w/w) soy protein with a mixed bacterial culture comprising, in addition to Lactobacillus delbrueckii, a strain capable of stimulating growth of Lactobacillus delbrueckii.

In a second aspect, the invention pertains to a fermented soy product comprising Lactobacillus delbrueckii and a strain capable of stimulating growth of Lactobacillus delbrueckii.

In a further aspect, the invention relates to a culture comprising Lactobacillus delbrueckii and a strain capable of stimulating growth of Lactobacillus delbrueckii.

In other aspects, the invention provides for the use of Lactobacillus plantarum B1839, B2482 and/or B2485 to stimulate growth of Lactobacillus delbrueckii, preferably Lactobacillus delbrueckii subsp. bulgaricus, preferably Lactobacillus delbrueckii subsp. bulgaricus ATCC-BAA365, ATCC-11842 or Ib, in an aqueous medium comprising 0.25-10% (w/w) soy protein, as well as its use in mixed culture fermentation of an aqueous medium comprising 0.25-10% (w/w) soy protein.

In yet another aspect, the invention sees to a method for identifying a lactic acid bacterial strain capable of stimulating growth of Lactobacillus delbrueckii in a medium, said method comprising the steps of: a) growing a monoculture of Lactobacillus delbrueckii in said medium; b) growing a mixed culture of Lactobacillus delbrueckii and said lactic acid bacterial strain in said medium; c) comparing the growth of Lactobacillus delbrueckii in said monoculture with that in said mixed culture; and d) selecting the lactic acid bacterial strain which increases colony forming units Lactobacillus delbrueckii in said medium.

### DETAILED DESCRIPTION OF EMBODIMENTS

Screening of the L. plantarum strains for mixed culture fermentation properties in soymilk yielded 40 L. plantarum-S. thermophilus cultures with high CFU levels of both species. This combination also dominated the 3-species mixed cultures including L. bulgaricus, however, L. bulgaricus was still detected in 5 of these cultures. Since L. bulgaricus was detected in all monoculture controls as well in the mixed culture controls with S. thermophilus it was concluded that growth of this species in mixed cultures with L. plantarum as well as in the 3-species cultures was L. plantarum strain dependent. Three of the L. plantarum strains, B1839, B2484, and B2485, even resulted in increased CFU levels of L. bulgaricus in the corresponding 3-species combinations. With L. plantarum strain B1839 the L. bulgaricus CFU level was highest: 171- and 23-fold higher than in the corresponding mono- and mixed culture controls, respectively. For this reason, strain B1839 was selected to study mono- and mixed-culture population dynamics during soymilk fermentation. In a batch fermenter L. bulgaricus levels remained below the detection limit (of 500 CFU/ml), but L. bulgaricus grew well in each of the mixed cultures. Also in the batch fermenter a substantial positive effect of L. plantarum B1839 and L. bulgaricus on each other was observed. Most likely the positive effect of L. bulgaricus on L. plantarum was related to proteolytic activity since strain B1839 was not able to degrade the available sucrose completely in the monoculture. Since L. bulgaricus only poorly ferments sucrose, we hypothesize that the stimulatory effect of L. plantarum on L. bulgaricus is related to release of sugar monomers from the soymilk sugars by L. plantarum.

In a first aspect, the present invention pertains to a method for preparing a fermented soy product, said method comprising the step of fermenting an aqueous medium comprising 0.25-10% (w/w) soy protein with a mixed bacterial culture comprising, in addition to Lactobacillus delbrueckii, a strain capable of stimulating growth of Lactobacillus delbrueckii.

The term “Lactobacillus delbrueckii” as used herein includes the subspecies Lactobacillus delbrueckii subsp. bulgaricus and Lactobacillus delbrueckii subsp. lactis.

The amount of soy protein in the fermented soy product according to the invention is 0.25-10% (w/w), preferably 0.5-10% (w/w), such as 0.5-8% (w/w), 0.5-7% (w/w), 1-7% (w/w), 2-6% (w/w), 3-6% (w/w), 4-6% (w/w). It is prepared using an aqueous medium comprising 0.25-10% (w/w), preferably 0.5-10% (w/w), 0.5-8% (w/w), such as 0.5-7% (w/w), 1-7% (w/w), 2-6% (w/w), 3-6% (w/w), 4-6% (w/w) soy protein. For example, such aqueous medium may be soy milk. Soy milk is a water extract of soy beans. Soy milk is generally made by grinding and heating soy beans, removing the fibrous okara (soy pulp), clarifying, and pasteurizing into a soy base. The term “soy protein” as used herein includes soy protein isolate, which may be (partially) hydrolysed, and soy protein concentrate, which may be (partially) hydrolysed.

Liquid soymilk, soy milk powder, soy protein isolate and soy protein concentate, and the like are commercially available, e.g. from Devansoy.

A mixed bacterial culture as used in the present invention comprises at least one Lactobacillus delbrueckii, preferably Lactobacillus delbrueckii subsp. bulgaricus, strain as well as at least one strain, preferably a lactic acid bacterial strain, capable of stimulating growth of Lactobacillus delbrueckii, preferably Lactobacillus delbrueckii subsp. bulgaricus. As used herein, the term “lactic acid bacteria” refers to gram-positive rod- or sphere-shaped bacteria that produce lactic acid as the principal or sole end product of carbohydrate fermentation. Well-known genera include Bifidobacterium, Carnobacterium, Enterococcus, Lactobacillus, Lactococcus, Lactospaera, Leuconostoc, Oenococcus, Pediococcus, Streptococcus, Vagococcus and Weissella. Said mixed bacterial culture may further comprise additional lactic acid bacteria, such as Streptococcus thermophilus, Lactobacillus acidophilus, Lactobacillus subsp. casei. In an embodiment, said mixed bacterial culture further comprises at least Streptococcus thermophilus. The mixed bacterial culture may also comprise non-lactic acid bacteria, e.g., bacteria of the genus Propionibacterium, or any other food grade bacteria.

Said Lactobacillus delbrueckii subsp. bulgaricus may be any Lactobacillus delbrueckii subsp. bulgaricus strain. The skilled person is capable of selecting Lactobacillus delbrueckii subsp. bulgaricus strains that may be used in the preparation of the fermented soy product of the present invention.

The fermentation of the aqueous medium comprising 0.25-10% (w/w) soy protein may include one or more of the following steps: a) providing said aqueous medium; b) subjecting said aqueous medium to thermal treatment and homogenisation; c) addition of said mixed bacterial culture; and d) allowing fermentation to take place at a temperature in the range of 33-45° C., preferably 35-43° C., even more preferably 37-40° C.

In an embodiment, said strain capable of stimulating growth of Lactobacillus delbrueckii, preferably Lactobacillus delbrueckii subsp. bulgaricus stimulates growth of L. delbrueckii, preferably Lactobacillus delbrueckii subsp. bulgaricus, in a sucrose-comprising aqueous medium, said medium preferably comprising sucrose and the oligosaccharides stachyose and raffinose as sole carbohydrates in addition to the 0.5-10% (w/w) soy protein. Thus, in a suitable embodiment, said aqueous medium further comprises sucrose, stachyose and/or raffinose as sole carbohydrates. Preferably, said sucrose is present in said aqueous medium in an amount of about 0.25 to about 20% (w/w), preferably 0.25-18% (w/w), 0.4-17% (w/w), 0.5-15% (w/w), 1-13% (w/w). “(w/w)”, as herein used, is an abbreviation for “by weight,” used to describe the concentration of a substance in a mixture or solution. As an example, a weight percentage of 2, 2% (w/w), means that the mass of the substance is 2% of the total mass of the aqueous medium, i.e. 2 g per 100 g aqueous medium.

In a suitable embodiment, said strain capable of stimulating growth of Lactobacillus delbrueckii, preferably Lactobacillus delbrueckii subsp. bulgaricus, is a Lactobacillus plantarum strain. Said Lactobacillus plantarum strain may be one or more strain selected from the group consisting of Lactobacillus plantarum B1839, B2484 and B2485. The Lactobacillus plantarum strain B1839 referred to herein corresponds to Lactobacillus plantarum SF2A35B published by Figuero et al. (1995. Lactic acid bacteria of the sour cassava starch fermentation. Lett. Appl. Microbiol. 21:126-130). It has been deposited at the Centraalbureau for Schimmelcultures under the Regulations of the Budapest Treaty, and received the deposit number CBS 125104. Lactobacillus plantarum strains B2484 (NCTH19-1) and B2485 (NCTH19-2) have been derived from sour pickled pork sausage from Vietnam (Nem Chua). “Nem chua” is made from minced pork, sliced pigskin and a mixture of powdered grilled rice, salt, pepper, sugar and garlic. These contents are mixed thoroughly before being wrapped with aromatic, fresh leaves into small, boxy rolls before being stored for the fermentation process for three to five days, and when it is ready it can be served direct or grilled on the charcoal stove. B2484 and B2485 have been isolated after the fermentation process, and prior to further processing. They have been deposited at the Centraalbureau for Schimmelcultures under the Regulations of the Budapest Treaty, and received the deposit numbers CBS 125105 (B2484) and CBS 125106 (B2485).

In a preferred embodiment, said strain capable of stimulating growth of Lactobacillus delbrueckii, preferably Lactobacillus delbrueckii subsp. bulgaricus, is Lactobacillus plantarum B1839, as the stimulatory effect on Lactobacillus delbrueckii subsp. bulgaricus in an aqueous medium comprising 0.25-10% (w/w) soy protein was most pronounced for this strain.

In an embodiment, said Lactobacillus delbrueckii subsp. bulgaricus is selected from the group consisting of Lactobacillus delbrueckii bulgaricus ATCC-BAA365, ATCC-11842, and Ib.

In a suitable embodiment, the fermented soy product is soy yogurt. The soy yogurt of the invention may be set-style yogurt with a firm gelled texture, or may be stirred-style yogurt with a spoonable or fluid texture. In an embodiment, the fermentation for the set-style soy yogurt takes place in the package of the yogurt after packaging. Stirred-type soy yogurt is preferably fermented in fermentation tanks. After fermentation, the product may be stirred at a low speed prior to its transport by pumping and filling. A moderate to low shear rate is critical to achieve the desired viscous properties of stirred-style yogurt. Sweeteners, salt and flavors, calcium, fruit and vitamins A and D may be added to the soy yogurt. Stabilizers may also be used in soy yogurt to improve the body and texture by increasing firmness, and, if fruit is included in the soy yogurt, helping to keep the fruit uniformly mixed in the soy yogurt. Stabilizers conventionally used in soy yogurt include alginates (carageenan), gelatins, gums (locust bean, guar), pectins, and starch. Further conventional soy yogurt ingredients may be used. One skilled in the art will be capable of determining suitable conventional soy yogurt ingredients.

In a further aspect, the present invention pertains to a fermented soy product comprising Lactobacillus delbrueckii, preferably Lactobacillus delbrueckii subsp. bulgaricus, and a strain capable of stimulating growth of Lactobacillus delbrueckii, preferably Lactobacillus delbrueckii subsp. bulgaricus.

Preferably, said strain capable of stimulating growth of Lactobacillus delbrueckii, preferably Lactobacillus delbrueckii subsp. bulgaricus, in particular in an aqueous medium comprising 0.25-10% (w/w) soy protein, is a Lactobacillus plantarum strain, preferably selected from the group consisting of Lactobacillus plantarum B1839, B2484, and B2485, more preferably Lactobacillus plantarum B1839.

In an embodiment, said Lactobacillus delbrueckii bulgaricus is selected from the group consisting of Lactobacillus delbrueckii bulgaricus ATCC-BAA365, ATCC-11842, and Ib. As outlined hereinabove, in an advantageous embodiment, the fermented soy product is soy yogurt.

The fermented soy product may comprise further food ingredients such as sugars, further proteins, fats and oils, fatty acids, stabilisers, thickeners, colourants, flavourants, and the like. It is within the skills of the skilled person to select such further food ingredients depending on the desired end product.

In yet another aspect, the invention is concerned with a mixed bacterial culture comprising Lactobacillus delbrueckii, preferably Lactobacillus delbrueckii subsp. bulgaricus, and a strain capable of stimulating growth of Lactobacillus delbrueckii, preferably Lactobacillus delbrueckii subsp. bulgaricus, in an aqueous medium comprising 0.25-10% (w/w) soy protein. The strain capable of stimulating growth of Lactobacillus delbrueckii, preferably Lactobacillus delbrueckii subsp. bulgaricus, in such medium may advantageously be a Lactobacillus plantarum strain, preferably selected from the group of Lactobacillus plantarum B1839, B2484, and B2485. More preferably, said strain capable of stimulating growth of Lactobacillus delbrueckii, preferably Lactobacillus delbrueckii subsp. bulgaricus, in such medium is Lactobacillus plantarum B1839. In a further embodiment, said Lactobacillus delbrueckii bulgaricus is selected from the group of Lactobacillus delbrueckii bulgaricus ATCC-BAA365, ATCC-11842 and Ib. The culture may be a yogurt starter culture, further comprising S. thermophilus. Alternatively, said strain capable of stimulating growth of Lactobacillus delbrueckii, preferably Lactobacillus delbrueckii subsp. bulgaricus, in an aqueous medium comprising 0.25-10% (w/w) soy protein may be added to the aqueous medium as (part of) an adjunct culture in addition to a starter culture comprising Lactobacillus delbrueckii, preferably Lactobacillus delbrueckii subsp. bulgaricus, and S. thermophilus.

The invention also relates to the use of Lactobacillus plantarum B1839, B2484, and/or B2485, to stimulate growth of Lactobacillus delbrueckii, preferably Lactobacillus delbrueckii subsp. bulgaricus, preferably Lactobacillus delbrueckii bulgaricus ATCC-BAA365, ATCC-11842 or Ib, preferably ATCC-BAA365, in an aqueous medium comprising 0.25-10% (w/w) soy protein.

The invention further pertains to the use of Lactobacillus plantarum B1839, B2484 and/or B2485, preferably B1839, in mixed culture fermentation of an aqueous medium comprising 0.25-10% (w/w) soy protein.

In yet another aspect, the present invention provides for a method for identifying a lactic acid bacterial strain capable of stimulating growth of Lactobacillus delbrueckii, preferably Lactobacillus delbrueckii subsp. bulgaricus, in a medium, said method comprising the steps of: i) growing a monoculture of Lactobacillus delbrueckii, preferably Lactobacillus delbrueckii subsp. bulgaricus, in said medium; ii) growing a mixed culture of Lactobacillus delbrueckii, preferably Lactobacillus delbrueckii subsp. bulgaricus, and said lactic acid bacterial strain in said medium; iii) comparing the growth of Lactobacillus delbrueckii, preferably Lactobacillus delbrueckii subsp. bulgaricus, in said monoculture with that in said mixed culture; and iv) selecting the lactic acid bacterial strain which increases colony forming units Lactobacillus delbrueckii, preferably Lactobacillus delbrueckii subsp. bulgaricus, in said medium. Said medium preferably is a sucrose-containing medium, more preferably an aqueous medium comprising 0.25-10% (w/w) soy protein, such as soy milk.

It will also be clear that the description, drawings and examples are included merely to illustrate some embodiments of the invention, and not to limit the scope of protection. Starting from this disclosure, many more embodiments will be evident to a skilled person, which are within the scope of protection and the essence of this invention and which are obvious combinations of prior art techniques and the disclosure of this patent.

### DRAWINGS

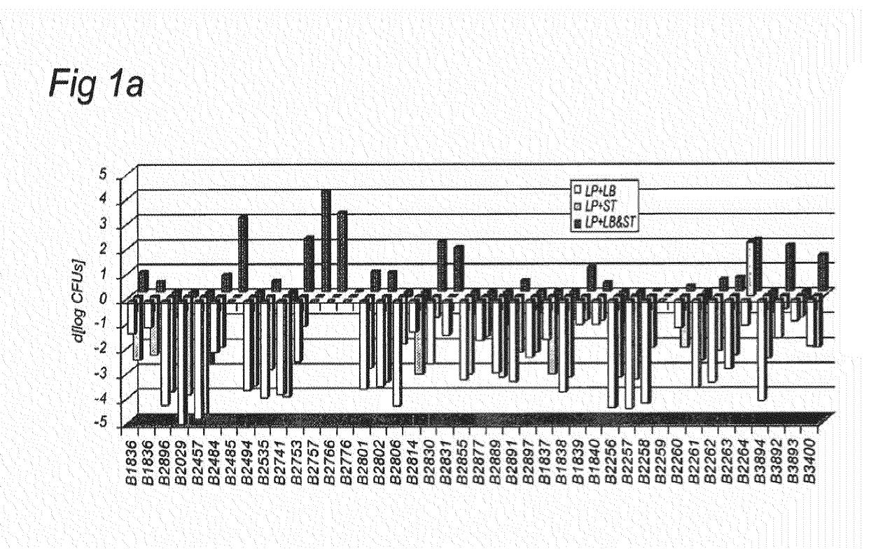

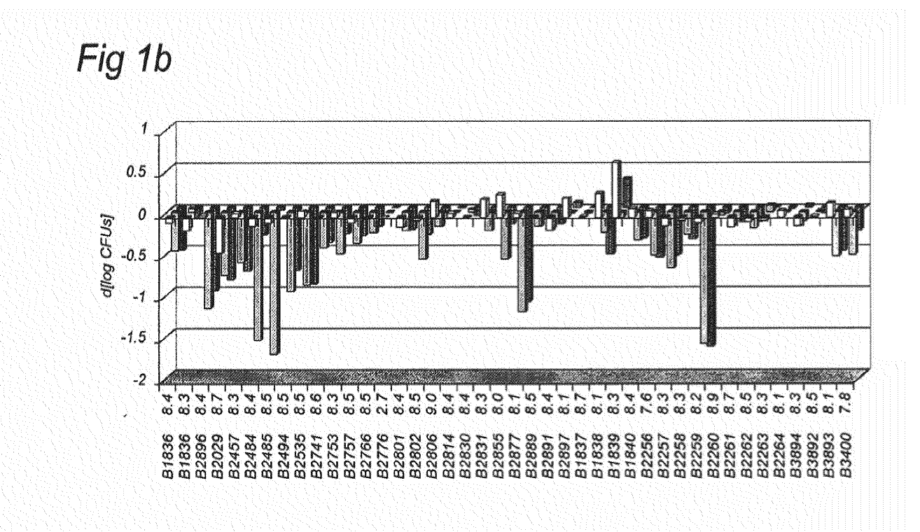

The invention is illustrated using FIG. 1, in which the effects of mixed culture growth on outgrowth of L. plantarum are compared to outgrowth in the corresponding mono-cultures, assessed for 41 individual L. plantarum strains. A. Milk fermentations, B. Soymilk fermentations. NIZO strain numbers and log CFU values for growth in monocultures are presented on the X-axis and the difference in outgrowth in the presence of S. thermophilus, L. bulgaricus or both is represented on the Y-axis (as difference in log₁₀ CFU values). Gray; mixed cultures with S. thermophilus, White; mixed cultures with L. bulgaricus, Dark grey; mixed cultures with both S. thermophilus and L. bulgaricus.

### EXAMPLES

### Materials and Methods

### Screening Approach

Mixed culture growth and acidification properties were studied in milk and in soy milk which were prepared by UHT treatment of reconstituted skimmed milk powder (Friesland Foods, Leeuwarden, the Netherlands) and Benesoy soy milk powder (Devansoy, Carroll, Iowa, USA). Lactobacillus delbrueckii subsp. bulgaricus strain ATCC-BAA365 and 41 Lactobacillus plantarum strains (Table 1) were cultured in MRS at 42 and 37° C., respectively, and Streptococcus thermophilus strain CNRZ1066 was cultured in GM17 at 42° C. Single and mixed cultures for fermentation of milk and soymilk were prepared in duplicate by diluting cultures of each strain to an O.D. (600 nm) of ˜1 and subsequently transferring 1% of the diluted culture to 10-ml polystyrene screw cap tubes containing 5-ml of freshly prepared substrate. Milk and soy milk cultures were incubated at 37° C. for 24 h and then transferred to fresh, preheated substrate using an inoculation needle. Before each transfer the cultures were mixed thoroughly for 15 min in an Infors Microtron II incubator (Infors AG, Switzerland). After 4 rounds of fermentation in milk or soy milk the cultures were examined for outgrowth of individual strains and end pH.

TABLE 1
The Lactobacillus plantarum strains.

| Strain | Source | Alternative name |
|---|---|---|
| B1836 | human D/S | WCFS1 |
| B2896 | vegetables | ATCC14917 |
| B2029 | Dairy | MLC43 |
| B2457 | fermented meat | CHEO3 |
| B2484 | fermented meat | NCTH19-1 |
| B2485 | fermented meat | NCTH19-2 |
| B2494 | fermented meat | NCTH27 |
| B2535 | fruit | LD2(2) |
| B2741 | vegetables | NOS140 |
| B2753 | Sourdough | Q2 |
| B2757 | Sourdough | H4 |
| B2766 | Sourdough | H14 |
| B2776 | Dairy | CECT4645 |
| B2801 | vegetables | KOG18 |
| B2802 | Dairy | KOG24 |
| B2806 | vegetables | LMG9208 |
| B2814 | fruit | Lp95 |
| B2830 | | |
| B2831 | Silage | |
| B2855 | fermented meat | N58 |
| B2877 | fermented meat | X17 |
| B2889 | fruit | LAC7 |
| B2891 | vegetables | LD3 |
| B2897 | vegetables | DKO22 |
| B1837 | Human C/F | 299 |
| B1838 | Human C/F | CIP104440 |
| B1839 | vegetables | SF2A35B |
| B1840 | vegetables | NCIMB12120 |
| B2256 | Human C/F | CIP104441 |
| B2257 | Human C/F | CIP104450 |
| B2258 | Human U | CIP104451 |
| B2259 | Human D/S | CIP104452 |
| B2260 | Human C/F | 299v, DSM9843 |
| B2261 | Silage | NC8 |
| B2262 | Silage | LM3 |
| B2263 | Silage | LP80 |
| B2264 | Silage | LP85-2 |
| B2726 | Silage | ATCC8014(2) |
| B3894 | vegetables | NCDO1193 |
| B3892 | Human D/S | CIP102359 |
| B3893 | Human D/S | CIP104448 |
| B3400 | Dairy | LMG18021 |

### Quantification of Growth; HT Plating

Colony forming units (CFU) were determined using a plating protocol based on the method described recently by Sieuwerts et al (LAM paper). Culture dilutions were prepared in TY broth using a multichannel pipette and were plated by pipetting 20 μl of each dilution on TY agar in 12 cm squared Petri dishes. L. plantarum plates contained 1% galactose and were incubated at 30° C. for 24-36 h. S. thermophilus was plated on TY agar of which the pH was adjusted to 7.5, and glucose (1%) was added as substrate. L. delbrueckii subsp. bulgaricus was plated on TY agar containing 1% mannose. For the (moderately thermophilic) yoghurt species the plates were first incubated at 37° C. for 4 h and then the temperature was increased to 45° C. L. delbrueckii subsp. bulgaricus plates were incubated under anoxic conditions in jars containing a mixture of nitrogen (85%), carbon dioxide (10%) and hydrogen (5%). CFU counts were determined after 24-36 h of incubation. End pH of the cultures was determined using hydroplates in a Tecan Safire II spectrophotometer (Tecan Austria GmbH, Austria).

### Population Dynamics

Population dynamics were studied in batch fermentors containing 1 L substrate from which samples were taken every 1.5 h. CFU's were determined according to the protocol described above and in addition samples were prepared for quantification by real-time quantitative PCR by mixing 0.1 ml sample with 0.9 ml TE. After mixing the samples, cell pellets were collected by centrifugation at 14,000×g and stored at −20° C. Primers for quantification of individual species based on 16S ribosomal DNA sequences were adapted from literature or designed using Amplicon and verified using Netprimer and Primer Express (Table 2). DNA was isolated using the Norgen Milk Bacterial DNA Isolation Kit. The amount of starting material was decreased to 0.1 ml and the digestion period increased to 1 h. Quantification was performed on an Applied Biosystems 7500 Fast detection system.

TABLE 2
Primers used for quantification of individual
species based on 16S ribosomal DNA sequences.
The DNA sequences of the primers used are
underlined, whereas the DNA sequence of the
entire amplified sequence is depicted.

L. delbrueckii bulgaricus strain ATCC-BAA-365
qPCR primer set Ldf4-Ldr4
CAAGTTTGAAAGGCGGCGTAAGCTGTCACTTTAGGATGAGCCCGCG
GCGCATTAGCTAGTTGGTGGGGTAAAGGCCTACCAAGGCAATGATG
CGTAGCCGAGTTGAGAGACTGATCGGCCA
L. plantarum WCFS1 qPCR primer set Lp2f-Lp2r
TGATCCTGGCTCAGGACGAACGCTGGCGGCGTGCCTAATACATGCA
AGTCGAACGAACTCTGGTATTGATTGGTGCTTGCA
S. thermophilus CNRZ1066 primer set Tf2-Tr2
AGGTAACCTGCCTTGTAGCGGGGGATAACTATTGGAAACGATAGCT
AATACCGCATAACAATGGATGACACATGTCATTTATTTGAAAGGGG
CAATTGCTCCACTACAAGATGGACCTGCGTTGTAT

### Results

### Combinatorial Screening Approach

Each of 41 L. plantarum strains was screened for the ability to ferment milk and soy milk in mixed culture combinations with S. thermophilus and L. delbrueckii subsp. bulgaricus and compared to growth in mono-cultures. The screening approach was developed to meet several criteria. To allow adaptation of each species to the fermentation conditions and the presence of one or more of the other species each culture was transferred 3 times to fresh substrate. This also increased the chance of selecting individuals with properties of interest since it enables fast growing strains to outcompete other strains for which no or only minor interdependencies exist. The fermentation temperature was set at 37° C. to allow good growth of each species, and before transferring to fresh substrate each culture was mixed thoroughly. To minimize the chance of (cross-)contamination, cultures were transferred using inoculation needles. To assess outgrowth of each species after 4 rounds of culturing in milk or soymilk a previously developed plating protocol (Sieuwerts, S., F. A. de Bok, E. Mols, W. M. de Vos, and J. E. van Hylckama Vlieg. 2008. A simple and fast method for determining colony forming units. Lett Appl Microbiol 47:275-8) was optimized for each of the 3 species of interest. Culture dilutions were prepared in TY broth using a multichannel pipette and 20 μl of each dilution was spotted using a multichannel pipette on 12 cm squared petridishes filled with TY agar, 6 spots per row and using only the odd or even tips of the pipette in order to use the available space optimally. TY agar was selected for plating because all 3 species and 41 L. plantarum strains produced CFU's on it which were comparable to growth on MRS agar (for L. delbrueckii and L. plantarum) or GM17 agar with 1% glucose (for S. thermophilus). L. delbrueckii and S. thermophilus plates were incubated at 45° C. to exclude growth of L. plantarum. However, because this resulted in significantly less CFU's of these species when compared to incubation at 37° C., the plates were first incubated at 37° C. for 4 h and then the temperature was increased to 45° C. Growth of L. delbrueckii on S. thermophilus plates was excluded by increasing the pH of the agar to 7.5, at which growth of S. thermophilus was not inhibited. Mannose was used as substrate for L. delbrueckii since API tests showed that S. thermophilus was not able to grow on it. Formation of colonies by L. delbrueckii was optimal under anaerobic conditions. L. plantarum plates contained galactose as selective substrate and these plates were incubated at 30° C. Culture dilutions were prepared in TY broth using a multichannel pipette and 20 μl of each dilution was spotted using a multichannel pipette on 12 cm squared Petri dishes filled with TY agar. For each culture dilutions 10⁻¹ to 10⁻⁵ were plated, by which the lower detection limit was set at 500 CFU's per ml. Agar plates, on which in total 36 culture dilutions were plated, were air dried and then incubated. End pH values of the cultures were determined using hydroplates, which were calibrated by including buffers within the range of pH 4 to 9.

### Screening of Mixed Cultures in Milk

A collection of 41 L. plantarum strains for which selection was based on differences in isolation source, geographic origin, and phylogenetic distance was screened for outgrowth in mono- and mixed culture milk fermentations with S. thermophilus and L. delbrueckii subsp. bulgaricus. After 4 rounds of culturing in milk viable cell counts in monocultures of 5 L. plantarum strains were below the detection limit of 500 CFU/ml, while most of the other strains produced at least more than 10⁶ CFU/ml under these conditions (FIG. 1). Although the CFU levels of 17 strains was more than 10⁷, none of the L. plantarum mono cultures in milk exceeded 10⁸ CFU/ml. None of the strains was thereby able to lower the pH of the milk below 6.3 which is only 0.25 less than the average pH of the milk controls. With one exception none of the L. plantarum strains produced more CFU when cultured together with either S. thermophilus or L. delbrueckii in milk. However, 21 strains produced more CFU when both S. thermophilus and L. bulgaricus were present, and nearly all of these strains showed poor growth in monocultures (FIG. 1). The presence of L. plantarum did not have a significant effect on outgrowth of S. thermophilus or L. bulgaricus in milk.

### Screening of Mixed Cultures in Soymilk

Screening for outgrowth of mono- and mixed cultures was also performed on soymilk using the same species, strains and combinations as described above for milk. With 1 exception all L. plantarum strains and S. thermophilus were able to ferment soymilk in monoculture. After 4 rounds of fermentation in soymilk L. bulgaricus was also still detected but did not exceed 10⁶ CFU/ml in any of the 5 monoculture controls (average; 3.1±1.7·10⁵) and thereby did not affect the pH. Outgrowth of several L. plantarum strains was reduced up to 2 log scales by the presence of S. thermophilus, while no substantial effect of L. plantarum on outgrowth of S. thermophilus was observed. Apart from a small stimulation of L. plantarum strain B1839 by L. bulgaricus, no substantial positive effects of L. bulgaricus, S. thermophilus or both on outgrowth on any of the L. plantarum strains was observed. The CFU levels of L. bulgaricus had dropped below the detection limit (500 CFU/ml) in nearly all 2- and 3-species combinations. However, in some combinations L. bulgaricus was still present and in the 2- and 3-species combinations with L. plantarum B1839, as well as in 3-species combinations with L. plantarum strains B2484 en B2485, its CFU levels were 10-100 times higher when compared to the corresponding cultures without L. plantarum. The levels in the co-culture with L. plantarum strain B2776 were comparable to the levels in L. bulgaricus monocultures, consistent with the inability of this strain to ferment soymilk.

### Population Dynamics of Mono- and Mixed Culture Milk and Soymilk Fermentations

Reproducibility of the screening experiment was assessed by repeating the screening protocol for several mono- and mixed cultures of interest with L. plantarum strains B1836 (WCFS1), B2485 and B1839 in soymilk and strains B2258 and B3400 in milk. The CFU-values for soymilk were comparable to the initial screening experiment, although L. bulgaricus produced 20 times more CFU in the mixed culture with strain B2485. For milk the results were comparable for strain B2258, but for outgrowth of B3400 in the 3-species mixed culture the CFU values were over 10-fold less. Strains B1839 and B3400 were selected for studying the population dynamics of soymilk and milk fermentations, respectively. The screening protocol was repeated for each mono- and mixed culture, but the 4^{th} fermentation was performed in batch (stirred) fermentors containing 1 L substrate from which samples were taken every 1.5 h. Growth of L. plantarum B3400 in milk under these conditions appeared to continue throughout the fermentation period with an intermediary lag phase between 4 and 12 h, and this pattern was similar for each of the 4 mixed culture combinations. Throughout the fermentation CFU values of B3400 for the 3-species fermentation were approximately 100 fold higher than the monoculture and between 10- and 1000-fold higher than each of the 2-species fermentations. The dynamics of S. thermophilus were nearly identical for the 4 cultures in which this species was present, and was characterized by 2 logarithmic growth phases. The dynamics of L. bulgaricus monoculture was nearly identical to the mixed culture with L. plantarum, while the dynamics of this species in the co culture with S. thermophilus was very similar to the 3-species milk fermentation.

In soymilk L. plantarum strain B1839 grew exponentially in the first 10-12 h of the fermentation irrespective of the presence of one or both of the other species, and in none of the cultures substantial increases in CFU were observed for the final 12 h of the fermentations. In the mixed culture with L. bulgaricus the CFU levels of B1839 were approximately 10-fold higher than for each of the other cultures throughout the fermentation period. In the mono-culture and the mixed culture with both yoghurt species CFU levels still increased after 10 h while the level in the culture with S. thermophilus remained fairly contant after 10 h of fermentation. The behavior of S. thermophilus was nearly identical in each of the soymilk cultures with an exponential growth phase in the first 6 h of fermentation and no substantial increase in CFU levels for the remainder of the fermentation period. The CFU level of L. bulgaricus in the mono-culture did not exceed the detection limit, while its dynamics in the mixed cultures with L. bulgaricus and both other species were comparable. A substantial positive effect of L. plantarum on L. bulgaricus was observed, in particular when S. thermophilus was not present. The experiments were repeated with 2 different L. delbrueckii subsp. bulgaricus strains, i.e., ATCC-11842 and Ib, with comparable results. Thus, it is concluded that L. plantarum B1839 has a positive effect on outgrowth of L. delbrueckii subsp. bulgaricus.

1. A method for preparing a fermented soy product, said method comprising: fermenting an aqueous medium comprising 0.25-10% (w/w) soy protein with a mixed bacterial culture comprising, in addition to Lactobacillus delbrueckii, a strain capable of stimulating growth of Lactobacillus delbrueckii.

2. The method according to claim 1, wherein said strain capable of stimulating growth of Lactobacillus delbrueckii is a Lactobacillus plantarum strain, optionally comprising Lactobacillus plantarum B1839, B2484 and/or B2485.

3. The method according to claim 1, wherein said Lactobacillus delbrueckii is Lactobacillus delbrueckii subsp. bulgaricus, optionally comprises Lactobacillus delbrueckii subsp. bulgaricus ATCC-BAA365, ATCC-11842 or Ib.

4. The method according to claim 1, wherein the fermented soy product comprises soy yogurt.

5. A fermented soy product comprising Lactobacillus delbrueckii and a strain capable of stimulating growth of Lactobacillus delbrueckii.

6. The fermented soy product according to claim 5, wherein said strain capable of stimulating growth of Lactobacillus delbrueckii is a Lactobacillus plantarum strain, optionally comprising Lactobacillus plantarum B1839, B2484, and/or B2485.

7. The fermented soy product according to claim 5, wherein said Lactobacillus delbrueckii is Lactobacillus delbrueckii subsp. bulgaricus, optionally comprises Lactobacillus delbrueckii subsp. bulgaricus ATCC-BAA365, ATCC-11842 or Ib.

8. The fermented soy product according to claim 5, wherein said soy product comprises an aqueous soy product comprising 0.25-10% (w/w) soy protein.

9. The fermented soy product according to claim 5, wherein said soy product comprises soy yogurt.

10. A culture comprising Lactobacillus delbrueckii and a strain capable of stimulating growth of Lactobacillus delbrueckii.

11. The culture according to claim 10, wherein said strain capable of stimulating growth of Lactobacillus delbrueckii is a Lactobacillus plantarum strain, optionally comprising Lactobacillus plantarum B1839, B2484 and/or B2485.

12. The culture according to claim 10, wherein said Lactobacillus delbrueckii bulgaricus is Lactobacillus delbrueckii subsp. bulgaricus, optionally comprising Lactobacillus delbrueckii subsp. bulgaricus ATCC-BAA365, ATCC-11842 or Ib.

13. A Lactobacillus plantarum B1839 capable of being used to stimulate growth of Lactobacillus delbrueckii, optionally comprising Lactobacillus delbrueckii subsp. bulgaricus, optionally comprising Lactobacillus delbrueckii subsp. bulgaricus ATCC-BAA365, ATCC-11842 or Ib., in an aqueous medium comprising 0.25-10% (w/w) soy protein.

14. A Lactobacillus plantarum B1839, B2484 and/or B2485, optionally comprising B 1839, capable of being used in mixed culture fermentation of an aqueous medium comprising 0.25-10% (w/w) soy protein.

15. A method for identifying a lactic acid bacterial strain capable of stimulating growth of Lactobacillus delbrueckii in a medium, said method comprising:
a) growing a monoculture of Lactobacillus delbrueckii in said medium;
b) growing a mixed culture of Lactobacillus delbrueckii and said lactic acid bacterial strain in said medium;
c) comparing growth of Lactobacillus delbrueckii in said monoculture with growth in said mixed culture; and
d) selecting a lactic acid bacterial strain which increases colony forming units Lactobacillus delbrueckii in said medium.

16. The method according to claim 15, wherein said medium is a sucrose-containing medium, optionally comprising an aqueous medium comprising 0.25-10% (w/w) soy protein, optionally comprising soy milk.


SEQUENCE LISTING (9 sequences)

SEQ ID NO: 1 - DNA, 20 bp, Artificial Sequence
  qPCR primer Ldf4
caagtttgaaaggcggcgta

SEQ ID NO: 2 - DNA, 20 bp, Artificial Sequence
  qPCR primer Ldr4
gttgagagactgatcggcca

SEQ ID NO: 3 - DNA, 20 bp, Artificial Sequence
  qPCR primer Lp2f
tgatcctggctcaggacgaa

SEQ ID NO: 4 - DNA, 21 bp, Artificial Sequence
  qPCR primer Lp2r
tggtattgattggtgcttgca

SEQ ID NO: 5 - DNA, 21 bp, Artificial Sequence
  qPCR primer Tf2
aggtaacctgccttgtagcgg

SEQ ID NO: 6 - DNA, 21 bp, Artificial Sequence
  qPCR primer Tr2
caagatggacctgcgttgtat

SEQ ID NO: 7 - DNA, 121 bp, Lactobacillus delbrueckii bulgaricus
  misc_feature (1)..(121): strain ATCC-BAA-365 amplified fragment
caagtttgaaaggcggcgtaagctgtcactttaggatgagcccgcggcgcattagctagttggtggggtaaaggcctaccaaggcaatgatgcgtagccgagttgagagactgatcggcca

SEQ ID NO: 8 - DNA, 81 bp, Lactobacillus plantarum
  misc_feature (1)..(81): strain WCFS1 amplified fragment
tgatcctggctcaggacgaacgctggcggcgtgcctaatacatgcaagtcgaacgaactctggtattgattggtgcttgca

SEQ ID NO: 9 - DNA, 127 bp, Streptococcus thermophilus
  misc_feature (1)..(127): strain CNRZ1066 amplified fragment
aggtaacctgccttgtagcgggggataactattggaaacgatagctaataccgcataacaatggatgacacatgtcatttatttgaaaggggcaattgctccactacaagatggacctgcgttgtat